(19) United States
(12) Patent Application Publication
(10) Pub. No.: US 2009/0058282 A1
(43) Pub. Date: Mar. 5, 2009

(54) Compound and Method of Using Same, Organic El Element, Method of Manufacturing Same, and Method of Using Same

(76) Inventors: Zhaomin Hou, Saitama (JP); Yu Liu, Saitama (JP)

Correspondence Address: NIXON & VANDERHYE, PC, 901 NORTH GLEBE ROAD, 11TH FLOOR, ARLINGTON, VA, 22203, US

(21) Appl. No.: 12/224,847

(22) PCT Filed: Nov. 28, 2006
(86) PCT No.: PCT/JP2006/323697

§ 371 Date: Sep. 8, 2008

(30) Foreign Application Priority Data
Mar. 10, 2006 (JP) 2006-065712

(51) Int. Cl. H01J 1/63; C07C 211/54; C07D 209/82

(52) U.S. Cl. 313/504; 564/372; 548/440

(57) ABSTRACT

The present invention relates to a compound of general formula (I) and a method of using the compound. The present invention relates also to an organic EL device including a pair of electrodes and a luminescent layer, sandwiched between the pair of electrodes, containing a luminescent material. The luminescent material contains the compound. The present invention relates also to a method of manufacturing the organic EL device using the compound and a method of using such an organic EL device. The compound provided by the present invention is a novel luminescent component which singly emits white light.

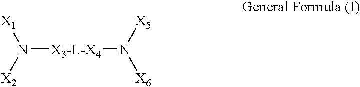

where each of X₁, X₂, X₅, and X₆ is independently an (un)substituted aryl group or monovalent aromatic heterocyclic group; X₁ and X₂ may be bonded to form a ring; X₅ and X₆ may be bonded to form a ring; each of X₃ and X₄ is independently an (un)substituted arylene group or divalent aromatic heterocyclic group; and L is either

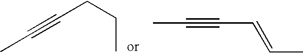

### TECHNICAL FIELD

The present invention relates to compounds suitable as luminescent material for use in an organic EL device and methods of using the compounds and also to organic EL devices containing such a compound and capable of producing white light, methods of manufacturing such a device, and methods of using such a device.

### BACKGROUND ART

Organic EL (electroluminescence) devices achieve high luminance at low power consumption, provide a long life, and have other advantages. For these reasons, the organic EL device have been increasingly applied to display devices and for illumination in recent years.

Known organic EL materials emit blue, red, or green light. Few materials are known which emit white light. Therefore, conventionally, a blue light emitting compound, a red light emitting compound, and a green light emitting compound were mixed to obtain a white light emitting organic EL device. In other words, the three colors were mixed to produce white color. See Appl. Phys. Lett. 1999, 74, 641-643, by Xie Z. Y., Huang J. S., Li Y. Q., and Shen J. C.; Appl. Phys. Lett. 1995, 67, 2281-2283, by Kido J., Shionoya H., and Nagai K.; and Angew. Chem. Int. Ed. 2002, 41, 182-184, by Liu Y., Guo J. H., Zhang H. D., and Wang Y.

### DISCLOSURE OF INVENTION

However, the production of white color using a mixture of a blue light emitting compound, a red light emitting compound, and a green light emitting compound requires adjustment of the mix ratio of the compounds in consideration of various factors including intensities of light produced by the compounds. The requirements inevitably lead to complex manufacturing steps, which are accompanied by reproducibility and stability issues.

Under these circumstances, the present invention has an objective of providing a novel compound that is singly capable of emitting white light.

The inventors of the present invention have diligently worked in order to accomplish the objective and as a result, found that particular conjugate enynes emit white light when a voltage is applied to them, which has led to the completion of the invention.

The present invention relates to a compound having general formula (I):

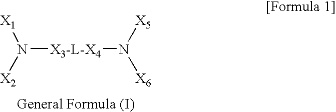

where each of X₁, X₂, X₅, and X₆ is independently an (un)substituted aryl group or monovalent aromatic heterocyclic group; X₁ and X₂ may be bonded to form a ring; X₅ and X₆ may be bonded to form a ring; each of X₃ and X₄ is independently an (un)substituted arylene group or divalent aromatic heterocyclic group; and L is either

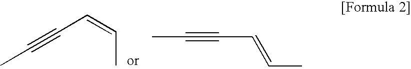

The compound preferably has general formula (II):

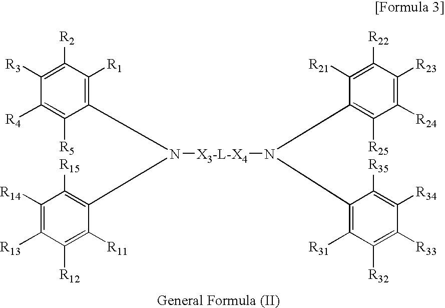

where each of R₁, R₂, R₃, R₄, R₅, R₁₁, R₁₂, R₁₃, R₁₄, R₁₅, R₂₁, R₂₂, R₂₃, R₂₄, R₂₅, R₃₁, R₃₂, R₃₃, R₃₄, and R₃₅ is independently a hydrogen atom, an (un)substituted alkyl group, an alkoxy group, or an aryl group; R₅ and R₁₅ may be bonded; R₂₅ and R₃₅ may be bonded; and L, X₃, and X₄ have the same definition as in general formula (I).

The compound more preferably has general formula (III):

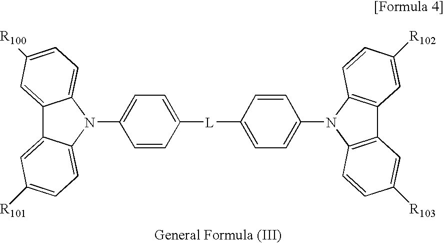

where each of R₁₀₀, R₁₀₁, R₁₀₂, and R₁₀₃ is independently an (un)substituted alkyl group, an alkoxy group, or an aryl group; and L has the same definition as in general formula (I).

The present invention relates also to an organic EL device including a pair of electrodes and a luminescent layer, sandwiched between the pair of electrodes, containing a luminescent material containing the compound of the present invention.

The present invention relates also to a method of using the organic EL device of the present invention as a white light emitter.

The present invention relates also to a method of manufacturing an organic EL device including a pair of electrodes and a luminescent layer, sandwiched between the pair of electrodes, containing a luminescent material, wherein the compound of the present invention is used as the luminescent material.

The present invention relates also to a method of using the compound of the present invention as a luminescent material in an organic EL device including a luminescent layer containing the luminescent material between a pair of electrodes.

The present invention realizes a white light emitting organic EL device using a single luminescent component.

### BEST MODE FOR CARRYING OUT INVENTION

The present invention will be described in more detail below.

The present invention relates to a compound having general formula (I):

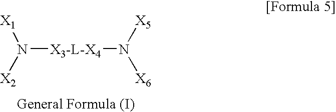

In general formula (I), each of X₁, X₂, X₅, and X₆ is independently an (un)substituted aryl group or monovalent aromatic heterocyclic group. Although X₁, X₂, X₅, and X₆ may be identical or different, they are preferably all identical for ease in synthesis and other merits. The aryl group has a carbon number of, for example, from 6 to 18, preferably from 6 to 14. Concrete examples of the aryl group include a phenyl group, a naphthyl group, and an anthracenyl group.

The monovalent aromatic heterocyclic group is, for example, a 5- or 6-membered aromatic heterocyclic group. Concrete examples include thiophene and pyridine. Each of X₁, X₂, X₅, and X₆ is preferably either a phenyl group or a naphthyl group to achieve good white light emission when used in an organic EL device. X₁ and X₂ may be bonded to form a ring, and X₅ and X₆ may be bonded to form a ring, in general formula (I). Especially, preferably, X₁ is bonded to X₂, and X₅ is bonded to X₆, to form carbazole rings.

When the aryl group or monovalent aromatic heterocyclic group is substituted, there are no particular limitations on the number, positions, and types of the substituents. Examples of the substituents include an alkyl group, an alkoxy group, and an aryl group. Concrete and preferred examples of the substituents will be given later in relation to R₁, etc. which appear in general formula (II). The substituents are preferably at ortho- or para-sites with respect to the N. The number of the substituents is preferably from 0 to 8, more preferably from 0 to 4.

In general formula (I), each of X₃ and X₄ is independently an (un)substituted arylene group or divalent aromatic heterocyclic group. Although X₃ and X₄ may be different, they are preferably identical for ease in synthesis and other merits. The arylene group has a carbon number of, for example, from 6 to 18, preferably from 6 to 14. Concrete examples of the arylene group include a phenylene group, a naphthylene group, and an anthracenylene group.

The divalent aromatic heterocyclic group is, for example, a 5- or 6-membered aromatic heterocyclic group. Concrete examples include thiophene and pyridine. X₃ and X₄ are preferably phenylene group to achieve good white light emission when used in an organic EL device.

When the arylene group or divalent aromatic heterocyclic group is substituted, there are no particular limitations on the number, positions, and types of the substituents. Preferable examples of the substituents include an alkyl group. Concrete and preferred examples of the substituents will be given later in relation to R₁, etc. which appear in general formula (II). The substituents are preferably at symmetric positions. The number of the substituents is preferably from 0 to 4, more preferably from 0 to 2.

The compound of the present invention contains the following linking group L which contains a double bond and a triple bond.

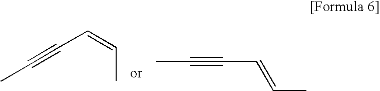

As explained above, the compound of the present invention has a conjugated enyne structure with both a double bond and a triple bond being present on the main chain and a π electron being delocalized along the main chain. The compound thus includes π electron conjugation throughout the molecule. For these reasons, the compound has unique optical functions and emits high luminance light when a voltage is applied to it.

In general formula (I), when L is

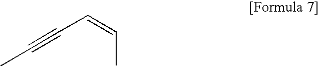

the compound is a cis-compound.

### When L is

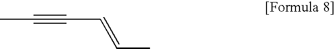

the compound is a trans-compound. The compound of the present invention, whether a cis-compound or a trans-compound, exhibits good luminance under voltage application. For luminescence stability, however, the compound preferably has a trans-configuration.

The compound of the present invention preferably has general formula (II):

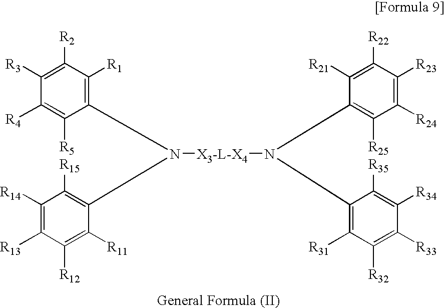

In general formula (II), each of R₁, R₂, R₃, R₄, R₅, R₁₁, R₁₂, R₁₃, R₁₄, R₁₅, R₂₁, R₂₂, R₂₃, R₂₄, R₂₅, R₃₁, R₃₂, R₃₃, R₃₄, and R₃₅ is independently a hydrogen atom, an (un)substituted alkyl group, an alkoxy group, or an aryl group. The alkyl group is an (un)branched alkyl group having a carbon number of, for example, from 1 to 20, preferably from 1 to 4. Concrete examples of the alkyl group include a methyl group, an ethyl group, a propyl group, an isopropyl group, and a t-butyl group. The alkoxy group is an (un)branched alkoxy group having a carbon number of from 1 to 8, preferably from 1 to 4. Concrete examples of the alkoxy group include a methoxy group, an ethoxy group, and a butoxy group. The aryl group has a carbon number of, for example, from 6 to 18, preferably from 6 to 14. Concrete examples of the aryl group include a phenyl group, a naphthyl group, and an anthracenyl group.

The compound of the present invention emits white light when used as a luminescent material in a luminescent layer of an organic EL device (detailed later). The light emitting property is adjustable through control of intermolecular distance in terms of the position and size of substituents. To achieve good white light emission, R₃, R₁₃, R₂₃, and R₃₃ preferably have a substituent. A preferable substituent is a t-butyl group.

In general formula (II), R₅ and R₁₅ may be bonded, and R₂₅ and R₃₅ may be bonded. Especially, preferably, R₅ is bonded to R₁₅, and R₂₅ is bonded to R₃₅, to form a carbazole ring with the benzene rings to which they are bonded.

In general formula (II), L, X₃, and X₄ have the same definition as in general formula (I). See the previous description in relation to general formula (I) for details.

The compound of the present invention more preferably has general formula (III):

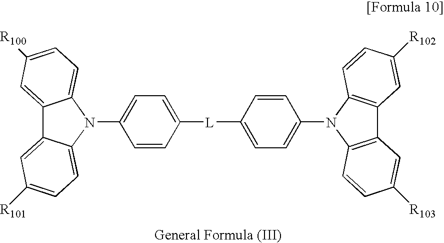

In general formula (III), each of R₁₀₀, R₁₀₁, R₁₀₂, and R₁₀₃ is independently an (un)substituted alkyl group, an alkoxy group, or an aryl group. See the previous description in relation to R₁, etc. which appear in general formula (II) for details. In general formula (III), L has the same definition as in general formula (I). See the previous description in relation to general formula (I) for details.

Concrete examples of the compound of the present invention are given below. Note however that the compound of the present invention is by no means limited to these concrete examples.

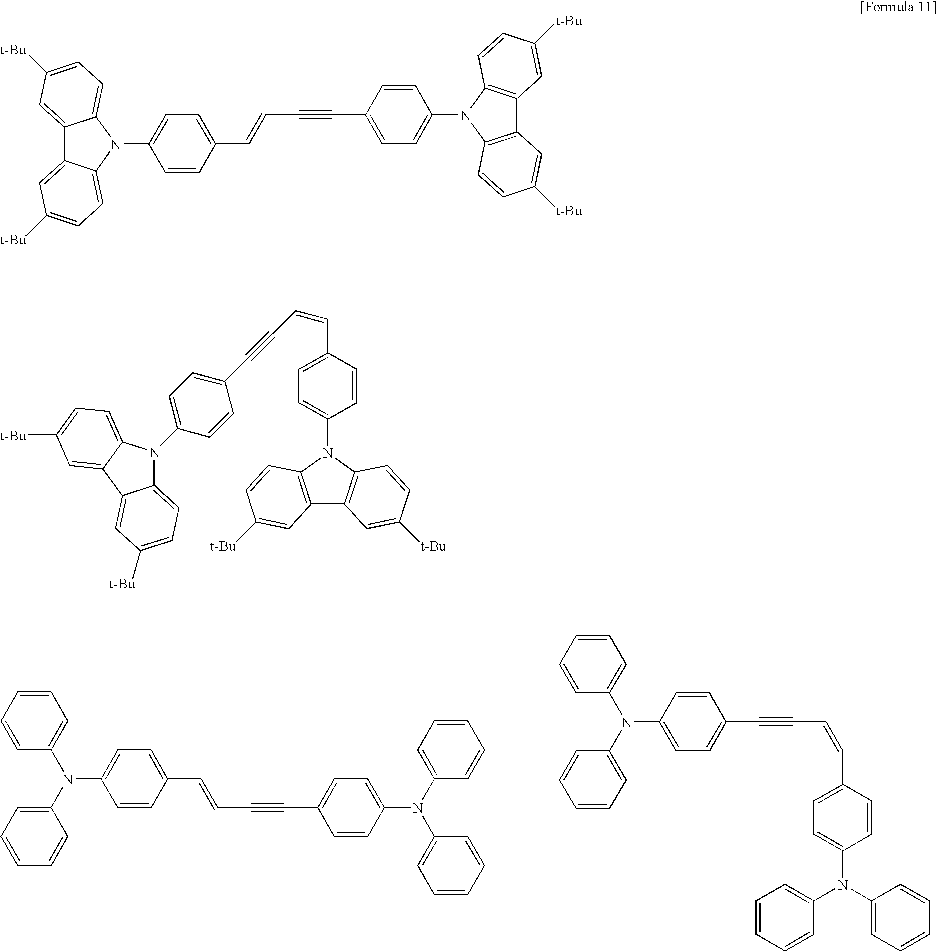

The compound of the present invention may be synthesized by any method. An example method is to dimerize the compound of Formula 13 which is the compound of Formula 12 having an ethinyl group introduced to an end.

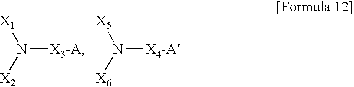

where A and A′ are a halogen atom, a hydroxyl group, or a like reactive group; and X₁, X₂, X₃, X₄, X₅, and X₆ have the same definition as given earlier.

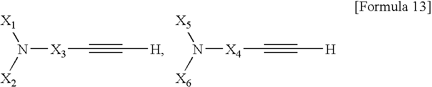

where X₁, X₂, X₃, X₄, X₅, and X₆ have the same definition as given earlier. The starting compounds can be synthesized by a known method. Some compounds are commercially available.

There are no particular limitations on the reaction conditions under which the dimerization may be carried out. The dimerization may be carried out, for example, in an inert atmosphere. If a solvent is to be used, there are no particular limitations on the type of the solvent. Suitable choices include aromatic hydrocarbons, such as benzene and toluene, and ethers, such as tetrahydrofuran and diethyl ether. Preferable examples include benzene, toluene, and other aromatic hydrocarbons.

The reaction temperature, although not limited in any particular manner, is, for example, from room temperature to the solvent's reflux temperature, preferably from room temperature (about 25° C.) to 110° C. The reaction time, although not limited in any particular manner, is, for example, from 15 minutes to 24 hours, preferably approximately from 15 minutes to 7 hours.

A catalyst with excellent stereoselectivity is preferably used to synthesize selectively either a cis- or a trans-compound in the dimerization. An exemplary catalyst with excellent cis-selectivity is the complex of general formula (IV):

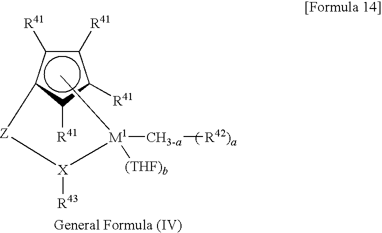

where each R⁴¹ is independently a hydrogen atom or an alkyl group; R⁴² is a trialkyl silyl group; “a” is either 1 or 2; THF is a tetrahydrofuran ligand; “b” is an integer from 0 to 2; M¹ is a rare-earth element, X is either a N or a P; R⁴³ is an aryl group or alkyl group with or without a substituent; and Z is a dialkyl silylene group.

In general formula (IV), The alkyl groups indicated by R⁴¹ are, for example, an (un)branched alkyl group having a carbon number of about 1 to 6, preferably of about 1 to 4. The alkyl groups indicated by R⁴¹, although possibly mutually different, are preferably all identical. Preferably, some of the alkyl groups indicated by R¹ are methyl groups. Most preferably, the alkyl groups are all methyl groups.

The alkyl group in the trialkyl silyl group indicated by R⁴² is the same as those indicated by R⁴¹. Examples of the trialkyl silyl group indicated by R⁴² include a trimethyl silyl group and a tert-butyl dimethyl silyl group. “a” is either 1 or 2, which is properly determined depending generally on the type of M¹, the properties of R⁴³, and other factors. The alkyl group in the dialkyl silylene group indicated by Z is again the same as those indicated by R⁴¹. The dialkyl silylene group indicated by Z is preferably, for example, a dimethyl silylene group.

In general formula (IV), M¹ is a rare-earth element, that is, one of scandium, yttrium, and 15 lanthanoids. Among them, ytterbium and lutetium are preferred. The most preferred is lutetium. In general formula (IV); the THF is a tetrahydrofuran ligand. The number of tetrahydrofuran ligands is from 0 to 2 and suitably selected depending on the type of M¹, the type of R₃, and other factors.

X is either a N (nitrogen atom) or a P (phosphorous atom). R⁴³ is an aryl group or alkyl group with or without a substituent. The aryl group indicated by R⁴³ is preferably a phenyl group. When the aryl group has a substituent or substituents, there are no particular limitations on the number, positions, and types of the substituents. Each or the substituent is preferably an alkyl group. When the aryl group is a phenyl group, the phenyl group preferably is not substituted or includes about 1 to 3 alkyl groups. The alkyl group indicated by R⁴³ is preferably unbranched, branched, cyclic, or a combination of these types, with a carbon number of about 1 to 12. The more preferred is a cyclic alkyl group having a carbon number of about 5 to 7.

When X is a N, R⁴³ may be an aryl group with or without a substituent, preferably a phenyl group with or without a substituent, such as an unsubstituted phenyl group, p-tolyl group, or a 2,6-dimethyl-4-tert-butyl phenyl group. When X is a P, R⁴³ may be an alkyl group with or without a substituent, preferably a cyclic alkyl group having a carbon number of about 5 to 7 with or without a substituent, for example, a cyclohexyl group.

An exemplary catalyst with excellent trans-selectivity preferably used in the dimerization is the complex of general formula (V):

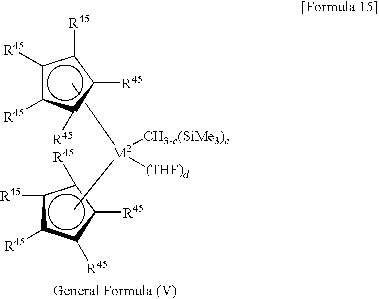

where each R⁴⁵ is independently a hydrogen atom, an alkyl group, or an aryl group with or without a substituent; “c” is either 1 or 2; THF is a tetrahydrofuran ligand; “d” is either 0 or 1; M² is lanthanum, cerium, praseodymium, neodymium, or samarium.

In general formula (V), the alkyl groups indicated by R⁴⁵ are, for example, an (un)branched alkyl group having a carbon number of about 1 to 6, preferably of about 1 to 4. The alkyl groups indicated by R⁴⁵, although possibly mutually different, are preferably all identical. Preferably, some of the alkyl groups indicated by R⁵ are methyl groups. Most preferably, the alkyl groups are all methyl groups. The aryl groups indicated by R⁴⁵ are, for example, preferably phenyl groups. When the aryl groups have a substituent or substituents, there are no particular limitations on the number, positions, and types of the substituents. Each or the substituent is preferably an alkyl group. When the aryl groups are phenyl groups, the phenyl groups preferably are not substituted or each include about 1 to 3 alkyl groups.

“c” is either 1 or 2, which generally relates to the THF, that is, the number “d” of tetrahydrofuran ligands. For example, when “c” is 2, “d” is 0; when “c” is 1, “d” is 1.

In general formula (V), M² is one of the rare-earth elements: lanthanum, cerium, praseodymium, neodymium, or samarium. Among them, lanthanum and cerium are preferred. The most preferred is lanthanum.

The complex of general formulae (IV) and (V) used as the catalyst may be used in any quantity: for example, about 0.001 to 0.10 mol, preferably about 0.02 to 0.05 mol, per mole of the compound having an ethinyl group introduced to an end (sum quantity if used with other compounds). The concentration in a reaction liquid may be, for example, about 0.001 to 1 mol/L, preferably about 0.01 to 0.1 mol/L. See Japanese Unexamined Patent Publication No. 2004-263072 (Tokukai 2004-263072; published) for the synthesis and other details of the complex of general formulae (IV) and (V). After the dimerization reaction, column chromatography and/or other known refinement processes may be carried out. Synthesis of a target product can be confirmed by, for example, mass spectroscopy, NMR, or another known method.

The compound of the present invention exhibits blue fluorescence in a solid state (cast film) and in a solution due to ultraviolet radiation. In contrast, when used as single luminescent component in a luminescent layer of an organic EL device, the compound emits white light. This is presumably because although the compound of the present invention itself emits blue light, when the compound is excited under an applied voltage to form an aggregate (excimer), a shift occurs to longer wavelengths due to intermolecular interaction. In other words, the aggregate after the shift to longer wavelengths contains an red light emitting excimer and a green light emitting excimer. Therefore, in the luminescent layer, the compound itself emits blue light and simultaneously, the excimer emits red and green light. As a result, the luminescent layer appears to emit white light. The luminescent layer has an advantage of excellent white light reproducibility and stability over a luminescent layer fabricated by mixing multiple materials which emit light of different colors. The compound of the present invention provides a good optically functional material if this characteristic light emitting property is exploited. The compound is suited for use in the organic EL device. The use of the compound in the organic EL device will be detailed later in reference to the organic EL device of the present invention.

The present invention relates also to an organic EL device including a pair of electrodes and a luminescent layer, sandwiched between the pair of electrodes, containing a luminescent material containing the compound of the present invention. The luminescent material may consist of the compound of the present invention. The luminescent layer emits high luminance white light when a voltage is applied across the pair of electrodes; without the need to use any other luminescent component than the compound of the present invention.

The present invention relates also to a method of manufacturing an organic EL device including a pair of electrodes and a luminescent layer, sandwiched between the pair of electrodes, containing a luminescent material, wherein the compound of the present invention is used as the luminescent material.

The present invention relates also to a method of using the organic EL device of the present invention as a white light emitter. Applying a voltage across the pair of electrodes causes the luminescent layer to emit white light.

The electrodes in the organic EL device of the present invention are an anode and a cathode. The anode, although not limited in particular manner, is normally transparent to allow the light emitted by the luminescent layer to escape. The transparent electrode is, for example, an ITO (Indium Tin Oxide) electrode. An ITO electrode is fabricated by, for example, coating a glass substrate with ITO by vacuum vapor deposition or another known method. ITO electrodes are also available on the market. The cathode, although not limited in particular manner, may be, for example, a lithium fluoride/aluminum electrode. The anode and cathode have a thickness, although not limited in particular manner, of, for example, from 50 to 400 nm, preferably from 100 to 300 nm. The anode and cathode has an electric resistance of, for example, from 5 to 50Ω, preferably from 10 to 30Ω.

The organic EL device of the present invention has a luminescent layer containing the compound of the present invention between the pair of electrodes. The luminescent layer may be formed by vacuum vapor deposition, film casting, or another known method. Considering operationality and other factors, vacuum vapor deposition is a preferred method.

The luminescent layer contains the compound of the present invention, and preferably consists of the compound of the present invention. The luminescent layer may contain other components which are normally used in the luminescent layer of organic EL devices. When the luminescent layer contains another component or components, the compound of the present invention preferably accounts for 95 mass % or more of the luminescent layer. The luminescent layer has a thickness of, for example, from 20 to 40 nm, preferably from 30 to 40 nm.

The organic EL device of the present invention may have any layer structure. For example, the anode, the luminescent layer, and the cathode may be stacked in this order. Alternatively, the anode, the hole injection layer, the luminescent layer, and the cathode may be stacked in this order. The organic EL device of the present invention is capable of emitting high luminance white light even with no hole injection layer. A hole injection layer may be provided between the anode and the luminescent layer for better light emission efficiency. The hole injection layer is not limited in any particular manner so long as it can inject holes from the anode when a voltage is applied. An example is a layer containing, as hole transport material, 4,4′-bis(1-naphthylphenylamino)biphenyl (NPB) or N,N-diphenyl-N,N′-bis m-tolylbenzidine (TPD). The hole injection layer may be formed by vacuum vapor deposition or another known film forming method.

The layer structure of the organic EL device of the present invention is by no means limited to the foregoing examples. Other examples are the anode, the hole injection layer, the luminescent layer, the electron transport layer, and the cathode stacked in this order, the anode, the hole injection layer, the luminescent layer, the hole blocking layer, the electron transport layer, and the cathode stacked in this order, and other known layer structures. In these cases, a known electron transport layer and hole blocking layer may be used.

The organic EL device of the present invention is capable of emitting white light when a voltage is applied. The emission has a peak at, for example, 400 to 700 nm, preferably 450 to 650 nm. The organic EL device of the present invention emits light, at a voltage of, for example, 30 V or lower, preferably from 5 to 20 V, more preferably from 7 to 15 V. The organic EL device of the present invention achieves luminance of, for example, 800 cd/m² or higher, preferably 1000 cd/m² or higher, more preferably 1300 cd/m² or higher, under 15-volts voltage application. Pure white is located at (0.33, 0.33) in CIE color coordinates. The organic EL device of the present invention is capable of emitting near pure white light.

The organic EL device of the present invention is suitable for use, for example, as a light source in the white light illumination device and also as a white light source in the organic EL display device, because of its capability to emit high luminance white light.

### EXAMPLES

The following will describe the present invention further by means of examples. The present invention is however by no means limited to these examples/embodiments.

### Synthesis Example 1

### 1. Synthesis of 3,6-di-tert-butyl-9H-carbazole

A three-neck flask was charged with 9-H-carbazole (3.3 g, 20 mmol), 100 mL of nitromethane, and ZnCl₂ (8.1 g, 60 mmol) in a nitrogen atmosphere. 2-chloro-2-methylpropane (6.5 mL, 60 mmol) was added dropwise while stirring. The mixture was stirred at room temperature for 5 hours. Next, 100 mL of water was added and the mixture was subjected to hydrolysis. The product was extracted with CH₂Cl₂ (3×60 mL). The organic layer was washed in water (2×150 mL) and dried with MgSO₄. Next, the product was placed in a vacuum for evaporation, to obtain 5.2 g (94%) of an off-white solid. The solid was subjected to mass spectroscopy and NMR to confirm that the solid was the target product.

MS: m/z 279 (M⁺). Anal. Calcd for C₂₀H₂₅N: C, 85.97; H, 9.02; N, 5.01. Found: C, 85.87; H, 8.94; N, 4.99. ¹H NMR (CDCl₃, 25° C., ppm): δ 8.07 (d, J=1.94 Hz, 2H), 7.83 (s b, 1H), 7.49 (dd, J=8.46, 1.93 Hz, 2H), 7.33 (dd, J=5.56, 0.49 Hz, 2H), 1.44 (s, 18H).

### Synthesis Example 2

### Synthesis of 9-(4-bromo-phenyl)-3,6-di-tert-butyl-9H-carbazole

o-dichlorobenzene (80 mL) containing the compound obtained in synthesis example 1 (4.2 g, 15 mmol), 1,4-dibromobenzene (11.8 g, 50 mmol), K₂CO₃ (8.3 g, 60 mmol), copper powder (0.64 g, 10 mmol), and 18-crown-6 (1.3 g, 5 mmol) was deaerated for 30 minutes in nitrogen while stirring. See Chem. Mater. 1540-1544, by Zhan X. W., Liu Y. Q., Zhu D. B., Huang W. T., and Gong Q. H. The reaction mixture was then subjected to refluxing for 12 hours in nitrogen. The unrefined mixture was filtered, and the residue was washed in CHCl₃ (3×10 mL). The collected filtrate was dried by evaporation. The residue was refined by flash column chromatography (silica gel, CH₂Cl₂ containing 50% hexane) to obtain a white solid (5.3 g, 82%). The solid was subjected to mass spectroscopy and NMR to confirm that the solid was the target product.

MS: m/z 433 (M⁺). Anal. Calcd for C₂₆H₂₈BrN: C, 71.89; H, 6.50; Br, 18.39; N, 3.22. Found: C, 71.72; H, 6.55; Br, 18.22N, 3.27. ¹H NMR (CDCl₃, 25° C., ppm): δ 8.12 (d, J=1.94 Hz, 2H), 7.69 (dd, J=6.33, 1.98 Hz, 2H), 7.45 (dd, J=9.10, 1.98 Hz, 2H), 7.43 (dd, J=6.73, 1.98 Hz, 2H), 7.30 (d, J=8.7 Hz, 2H), 1.46 (s, 18H); ¹³C NMR (CDCl₃, 25° C., ppm): δ 31.98, 34.70, 108.99, 116.30, 120.20, 123.61, 125.82, 128.21, 132.94, 137.24, 138.91, 143.12.

### Synthesis Example 3

### Synthesis of 3,6-di-tert-butyl-9-(4-ethinyl-phenyl)-9H-carbazole

2-methyl-3-butyn-2-ol (1.26 g, 15 mmol) was added to dry piperidine (30 mL) containing the compound obtained in synthesis example 2 (4.3 g, 10 mmol). This solution was deaerated for 30 minutes in nitrogen while stirring. Thereafter, Pd(PPh₃)₂Cl₂ (70 mg, 0.1 mmol) and CuI (47.5 mg, 0.25 mmol) were added to the solution. Next, the reaction mixture was subjected to refluxing for 10 hours in nitrogen. After the reaction is completed, the unrefined mixture was filtered at room temperature. The precipitate was washed in flowing diethyl ether. The collected filtrate was dried by evaporation. Next, the residue was dissolved in 50 mL of 2-propanol, and KOH powder (1.5 g, 25 mmol) was added. See Org. Lett. 2001, 3, 2005-2007, by Lee S. H., Nakamura T., and Tsutsui T.; and Synthesis 1980, 627-630, by Takahashi S., Kuroyama Y., Sonogashira K., and Hagihara N. The mixture was subjected to refluxing for 2 hours in nitrogen while stirring rigorously. After hydrolysis, the product was extracted with diethyl ether and dried with MgSO₄ then by evaporation. The residue was refined by flash column chromatography (silica gel, petroleum ether containing 10% ethyl acetate) to obtain a white solid (2.65 g, 70%). The solid was subjected to mass spectroscopy and NMR to confirm that the solid was the target product.

MS: m/z 379 (M⁺). Anal. Calcd for C₂₈H₂₉N: C, 88.61; H, 7.70; N, 3.69. Found: C, 88.45; H, 7.56; N, 3.76. ¹H NMR (CDCl₃, 25° C., ppm): δ 8.15 (d, J=1.93 Hz, 2H), 7.68 (d, J=8.7 Hz, 2H), 7.51 (d, J=8.7 Hz, 2H), 7.45 (dd, J=8.69, 1.94 Hz, 2H), 7.35 (d, J=8.7 Hz, 2H), 3.14 (s, 1H), 1.46 (s, 18H); ¹³C NMR (CDCl₃, 25° C., ppm): δ 31.98, 34.71, 77.90, 83.07, 109.15, 116.28, 120.20, 123.59, 126.29, 126.29, 133.56, 138.61, 143.21.

The scheme for the above reaction is shown below.

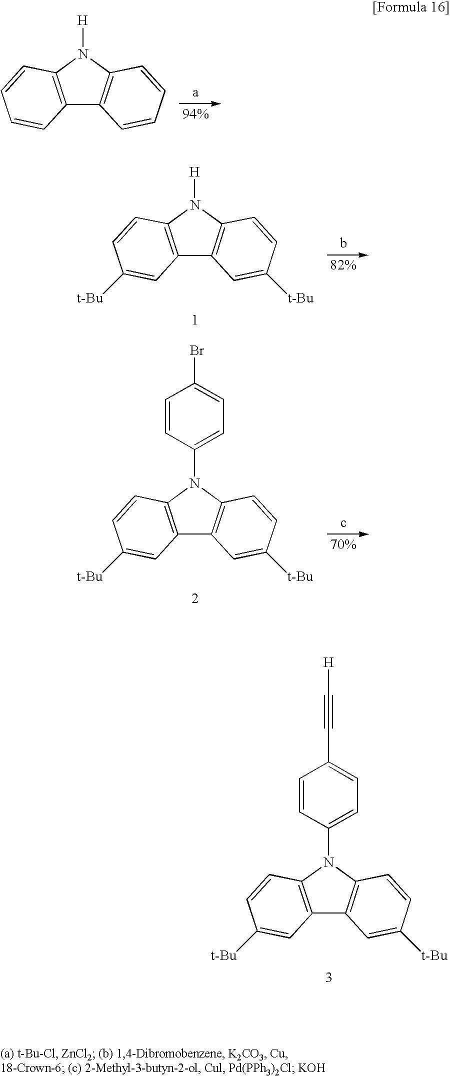

### Synthesis Example 4

### Synthesis of 3,6-di-tert-butyl-9-(4-[4-[4-(3,6-di-tert-butyl-carbazol-9-yl)-phenyl]-Z-but-1-en-3-inyl]-phenyl) (cis-compound)

In a glove box, a benzene solution (3 mL) of catalyst 1 (0.038 g, 0.05 mmol) illustrated below was added to a benzene solution of the compound (0.38 g, 1 mmol) obtained in synthesis example 3 placed in a Schlenk tube. See J. Am. Chem. Soc. 2003, 125, 1184-1185, by Nishiura M., Hou Z., Wakatsuki Y., Yamaki T., and Miyamoto T. The tube was taken out and the mixture was stirred for 5 hours at 80° C. (oil bath). As the reaction mixture was cooled down to room temperature, a yellow precipitate formed. After the benzene solvent was removed at reduced pressure, the residue was refined by flash column chromatography (silica gel, CH₂Cl₂) to obtain a yellow solid (0.37 g, 98%). This solid was subjected to mass spectroscopy and NMR to confirm that the target product, a cis-compound, was obtained at a selectivity in excess of 99%. The cis-selectivity was calculated from the integral ratio of peaks in the ¹H NMR and ¹³C NMR analyses.

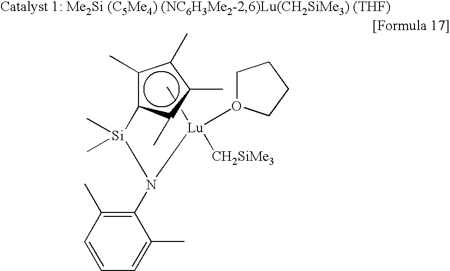

MS: m/z 758 (M⁺). Anal. Calcd for C₅₆H₅₈N₂: C, 88.61; H, 7.70; N, 3.69. Found: C, 88.49; H, 7.44; N, 3.86. ¹H NMR (CDCl₃, 25° C., ppm): δ 8.18 (d, J=8.46 Hz, 2H), 8.13 (s, 4H), 7.70 (d, J=8.7 Hz, 2H), 7.62 (d, J=8.7 Hz, 2H), 7.56 (d, J=8.7 Hz, 2H), 7.48-7.36 (m, 8H), 6.83 (d, J=12.80 Hz, 1H), 6.04 (d, J=11.84 Hz), 1.46 (s, 36H); ¹³C NMR (CDCl₃, 25° C., ppm): δ 32.07, 34.81, 88.87, 95.90, 107.56, 109.18, 116.20, 121.45, 123.49, 123.56, 126.12, 126.34, 130.01, 132.75, 134.91, 137.80, 138.10, 138.24, 138.75, 143.02.

### Synthesis Example 5

### Synthesis of 3,6-di-tert-butyl-9-(4-[4-[4-(3,6-di-tert-butyl-carbazol-9-yl)-phenyl]-E-but-1-en-3-inyl]-phenyl) (trans-compound)

In a glove box, a toluene solution (3 mL) of catalyst 2 (0.03 g, 0.05 mmol) illustrated below was added to a toluene solution (2 mL) of the compound (0.38 g, 1 mmol) obtained in synthesis example 3 placed in a flask, while stirring rigorously for 5 minutes. As a result, a yellow precipitate formed. See Organometallics 1991, 10, 1980-1986, by Heeres H. J. and Teuben J. H. After the toluene solvent was removed at reduced pressure, the residue was refined by flash column chromatography (silica gel, CH₂Cl₂) to obtain a yellow solid (0.37 g, 98%). This solid was subjected to mass spectroscopy and NMR to confirm that the target product, a trans-compound, was obtained at a selectivity in excess of 99%.

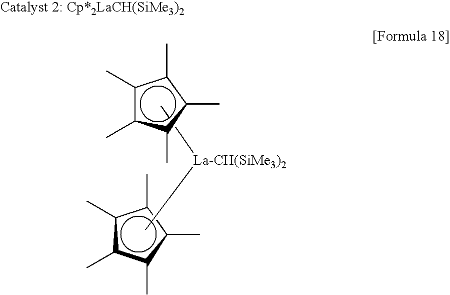

MS: m/z 758 (M⁺). Anal. Calcd for C₅₆H₅₈N₂: C, 88.61; H, 7.70; N, 3.69. Found: C, 88.40; H, 7.49; N, 3.81. ¹H NMR (CDCl₃, 25° C., ppm): δ 8.13 (s, 4H), 7.70 (d, J=8.46 Hz, 2H), 7.65 (d, J=8.46 Hz, 2H), 7.56 (d, J=8.46 Hz, 2H), 7.56 (d, J=8.46 Hz, 2H), 7.47 (d, J=8.7 Hz, 4H), 7.39 (d, J=8.7 Hz, 4H), 7.18 (d, J=16.19 Hz, 1H), 6.51 (d, J=15.94 Hz, 1H), 1.47 (s, 36H); ¹³C NMR (CDCl₃, 25° C., ppm): δ 31.99, 34.74, 89.63, 91.72, 108.45, 109.22, 116.28, 121.70, 123.57, 123.70, 126.38, 126.70, 127.61, 132.94, 134.74, 138.11, 138.48, 138.91, 140.53, 143.14.

The scheme for the above reaction is shown below.

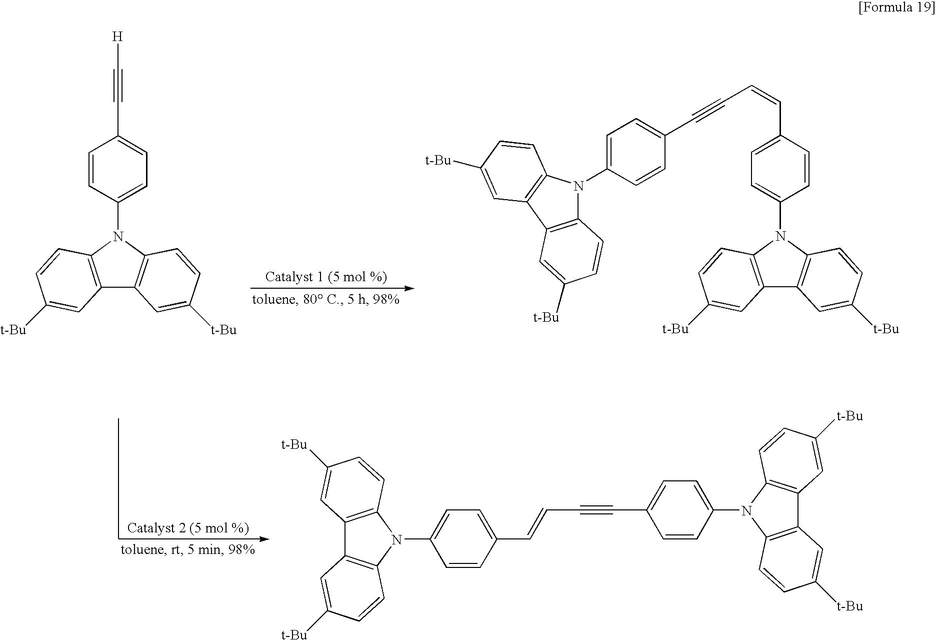

### Synthesis Example 6

### Synthesis of (4-ethinyl-phenyl)diphenylamine

(4-ethinyl-phenyl)diphenylamine was synthesized by the following reaction scheme.

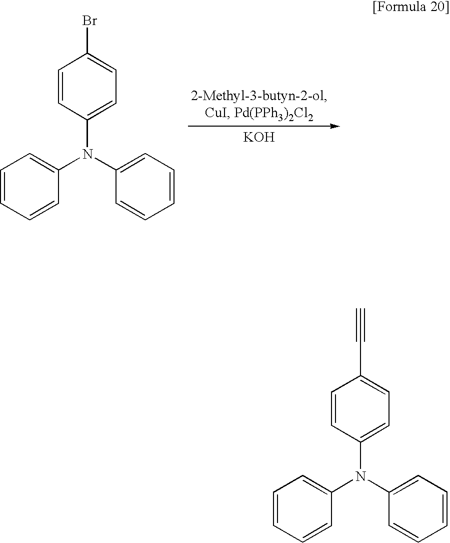

First, 2-methyl-3-butyn-2-ol (1.26 g, 15 mmol) was added to dry piperidine (30 mL) containing a commercially available starting compound (3.23 g, 10 mmol). This solution was deaerated for 30 minutes in nitrogen while stirring. Thereafter, Pd(PPh₃)₂Cl₂ (70 mg, 0.1 mmol) and CuI (47.5 mg, 0.25 mmol) were added to the solution. Next, the reaction mixture was subjected to refluxing for 10 hours in nitrogen. After the reaction is completed, the unrefined mixture was filtered at room temperature. The precipitate was washed in flowing diethyl ether. The collected filtrate was dried by evaporation. Next, the residue was dissolved in 50 mL of 2-propanol, and KOH powder (1.5 g, 25 mmol) was added. The mixture was subjected to refluxing for 2 hours in nitrogen while stirring rigorously. After hydrolysis, the product was extracted with diethyl ether and dried with MgSO₄ then by evaporation. The residue was refined by flash column chromatography (silica gel, petroleum ether containing 10% ethyl acetate) to obtain a white solid (1.88 g, 70%). The solid was subjected to mass spectroscopy and NMR to confirm that the solid was the target product.

¹H NMR (CDCl₃, 25° C., ppm): δ 7.29 (d, J=8.70 Hz, 2H), 7.22 (t, J=7.49 Hz, 4H), 7.07 (d, J=7.85 Hz, 4H), 7.02 (t, J=7.37 Hz, 2H), 6.94 (d, J=8.70 Hz, 2H), 2.96 (s, 1H); ¹³C NMR (CDCl₃, 25° C., ppm): δ 76.25, 83.84, 114.68, 121.91, 123.46, 124.86, 129.25, 132.88, 146.91, 148.11.

### Synthesis Example 7

### Synthesis of bis(4,4-triphenylamine)-Z-buten-3-inyl (cis-compound)

In a glove box, a benzene solution (3 mL) of catalyst 1 (0.038 g, 0.05 mmol) was added to a benzene solution of the compound (0.27 g, 1 mmol) obtained in synthesis example 6 placed in a Schlenk tube. The tube was taken out and the mixture was stirred for 5 hours at 80° C. (oil bath). As the reaction mixture was cooled down to room temperature, a yellow precipitate formed. After the benzene solvent was removed at reduced pressure, the residue was refined by flash column chromatography (silica gel, CH₂Cl₂) to obtain a yellow solid (0.26 g, 98%). This solid was subjected to mass spectroscopy and NMR to confirm that the target product, a cis-compound was obtained at a selectivity in excess of 99%. The cis-selectivity was calculated from the integral ratio of peaks in the ¹H NMR and ¹³C NMR analyses. FIG. 9 shows a photoluminescence spectrum of the cis-compound obtained in synthesis example 7 in a solution state (10⁻⁵ mol/LCH₂Cl₂ solution).

¹H NMR (CDCl₃, 25° C., ppm): δ 7.74 (d, J=8.94 Hz, 2H), 7.15-7.26 (m, 10H), 6.92-7.04 (m, 14H), 6.88 (d, J=8.94 Hz, 2H), 6.48 (d, J=11.84 Hz, 1H), 5.70 (d, J=11.84 Hz, 1H); ¹³C NMR (CDCl₃, 25° C., ppm): δ 88.13, 96.39, 105.35, 116.31, 122.21, 122.44, 123.26, 123.56, 124.80, 125.00, 129.30, 129.38, 129.68, 130.82, 132.26, 137.17, 147.11, 147.38, 147.83, 147.87.

### Synthesis Example 8

### Synthesis of bis(4,4-triphenylamine)-Z-buten-3-inyl (trans-compound)

In a glove box, a toluene solution (3 mL) of catalyst 2 (0.03 g, 0.05 mmol) was added to a toluene solution (2 mL) of the compound (0.27 g, 1 mmol) obtained in synthesis example 6 placed in a flask, while stirring rigorously for 5 minutes. As a result, a yellow precipitate formed. See Organometallics 1991, 10, 1980-1986, by Heeres H. J. and Teuben J. H. After the toluene solvent was removed at reduced pressure, the residue was refined by flash column chromatography (silica gel, CH₂Cl₂) to obtain a yellow solid (0.26 g, 98%). This solid was subjected to mass spectroscopy and NMR to confirm that the target product, a trans-compound, was obtained at a selectivity in excess of 99%. FIG. 10 shows a photoluminescence spectrum of the trans-compound obtained in synthesis example 8 in a solution state (10⁻⁵ mol/LCH₂Cl₂ solution).

¹H NMR (CDCl₃, 25° C., ppm): δ 7.24-7.31 (m, 12H), 6.96-7.11 (m, 16H), 6.92 (d, J=16.19 Hz, 1H), 6.24 (d, J=16.19 Hz, 1H); ¹³C NMR (CDCl₃, 25° C., ppm): δ 88.75, 91.74, 106.18, 116.49, 122.33, 122.89, 123.30, 123.46, 124.75, 124.92, 127.08, 129.31, 129.35, 130.36, 132.33, 139.94, 147.18, 147.30, 147.65, 148.10.

The scheme for the above reaction is shown below.

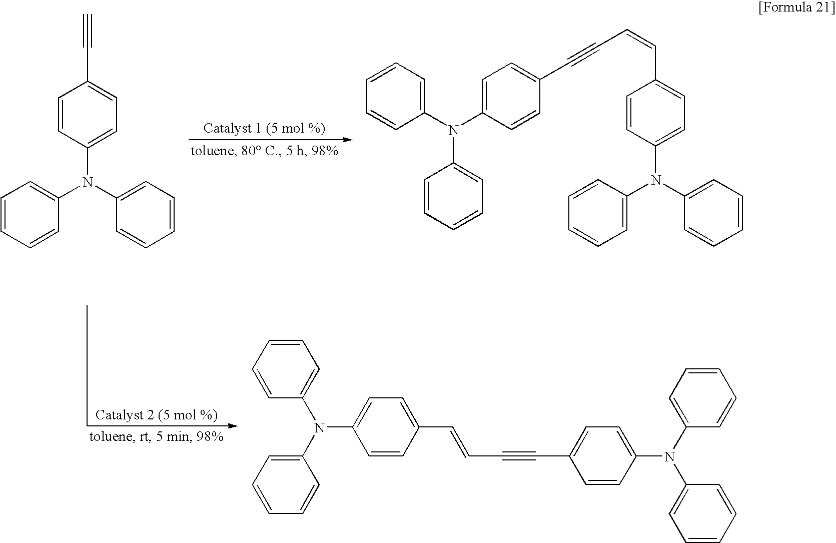

### Example 1

### Characterization of Luminescence of Cis-Compound and Trans-Compound

FIG. 1 shows an ultraviolet-to-visible absorption spectrum and a photoluminescence spectrum of the cis-compound obtained in synthesis example 4 in a solution state (10⁻⁵ mol/LCH₂Cl₂ solution) and in a solid state (60 nm vacuum vapor deposition film). FIG. 2 shows an ultraviolet-to-visible absorption spectrum and a photoluminescence spectrum of the trans-compound obtained in synthesis example 5 in a solution state (10⁻⁵ mol/LCH₂Cl₂ solution) and in a solid state (60 nm vacuum vapor deposition film).

FIGS. 1 and 2 show no significant difference in absorption pattern between the solid and solution states. That suggests that the cis-compound and the trans-compound had substantially the same ground state both in the solid state and the solution state and also that no appreciable intermolecular interaction existed in the solid state when the compounds were in ground state. In solution state, when under ultraviolet light (365 nm), both the cis-compound and the trans-compound exhibited intense blue fluorescence with relatively sharp emission peaks (λmax=455 nm, emission quantum yield=70% for cis-compound and 75% for trans-compound). However, the photoluminescence (PL) spectrum for the solid state indicates strong emission at 455 nm and moderate emission in a very wide range from 510 to 520 nm which tails off toward 600 nm. The phenomenon was presumably because a small amount of aggregate (excimer) was formed. See Macromolecules 2002, 35, 1988-1990 by Zhang Z.-B., Fujiki M., Tang H.-Z., Motonaga M., and Torimitsu K; and Macromolecules 2004, 37, 7578-7583 by Takihana Y., Shiotsuki M., Sanda F., and Masuda T.

### Example 2

### Fabrication of Organic EL Device 1

A commercially available anode was used. The anode was made of 2-mm thick glass substrate and an ITO film provided on the substrate. The anode had a sheet resistance of 200/cm⁻². The anode was washed by supersonic treatment in a solution of a cleaning agent, washed in flowing acetone, boiled in isopropanol, and washed in flowing methanol and then in flowing deionized water. After each washing step, the anode was dried in a vacuum oven. Thereafter, the trans-compound obtained in synthesis example 5 and a lithium fluoride/aluminum alloy were vacuum-vapor-deposited in this order on the ITO film to complete the fabrication of an organic EL device (“organic EL device 1”). Before the vapor deposition, vapor deposition material had been refined by sublimation. The luminescent layer of the cis-compound had a thickness of 60 nm, and the cathode of the lithium fluoride/aluminum alloy had a thickness of 200 nm.

### Example 3

### Fabrication of Organic EL Device 2

An organic EL device (“organic EL device 2”) was fabricated by the same process as in example 2, except that the luminescent layer was formed from the cis-compound synthesized in synthesis example 4.

### Example 4

### Fabrication of Organic EL Device 3

An organic EL device (“organic EL device 3”) was fabricated by the same process as in example 2, except that the thickness of the luminescent layer was altered to 40 nm and also that a hole injection layer was formed by vapor depositing 4,4′-bis(1-naphthylphenylamino)biphenyl (NPB) to a thickness of 30 nm between the anode and the luminescent layer.

### Example 5

### Fabrication of Organic EL Device 4

An organic EL device (“organic EL device 4”) was fabricated by the same process as in example 3, except that the thickness of the luminescent layer was altered to 40 nm and also that a hole injection layer was formed by vapor depositing 4,4′-bis(1-naphthylphenylamino)biphenyl (NPB) to a thickness of 30 nm between the anode and the luminescent layer.

FIG. 3 is schematics of organic EL devices 1 to 4.

Evaluation of Organic EL Devices

The EL spectrum, luminance, CIE color coordinates, and current-voltage-luminance properties of organic EL devices 1 to 4 were measured using a computer-controlled, programmable, direct-current (DC) source and a high speed scanning system incorporating a Photo Research PR650 spectrophotometer. FIG. 4 shows EL spectra under applied voltage. The luminance-voltage and current-voltage properties were measured at room temperature in the air. Results are shown in Table 1. The current-voltage-luminance properties are shown in FIG. 5 (organic EL device 1), FIG. 6 (organic EL device 2), FIG. 7 (organic EL device 3), and FIG. 8 (organic EL device 4).

TABLE 1

| | Emission | 1931 CIE under | | Max. | |
|---|---|---|---|---|---|
| | starting | applied voltage | | current | Max. |
| | voltage | 15 V | | efficiency | brightness |
| | (V) | X | Y | (cd/A) | (cd/m²) |
| Device 1 | 9.5 | 0.29 | 0.30 | 0.57 | 422 |
| Device 2 | 9 | 0.23 | 0.26 | 0.54 | 545 |
| Device 3 | 7 | 0.32 | 0.33 | 2.07 | 1395 |
| Device 4 | 7.5 | 0.24 | 0.28 | 1.64 | 1071 |

The EL spectra of organic EL devices 1 and 2 shown in FIG. 4 had noticeably broader peaks and more intense emission at long wavelengths around 510 to 590 nm than the PL spectra in the solid state shown in FIGS. 1 and 2. The phenomenon was presumably because aggregate (excimer) was formed under applied voltage. As a voltage was applied across the electrodes in organic EL devices 1 to 4 and gradually increased, the luminescent layer started to emit intense white light at a certain voltage (“emission starting voltage” in Table 1). Organic EL devices 1 to 4 emitted white light presumably due to the blue light emission (about 455 nm) by the compound itself and the green light emission (about 510 nm) and orange-red light emission (about 590 nm) at relatively long wavelengths by the excimer as illustrated in FIG. 4. Attention should be paid to organic EL device 3 which achieved a luminance as high as 1395 cd/m² at 16.5 V. This figure is better than those achieved by any other currently known organic EL devices made of luminescent material that is singly capable of emitting light. Organic EL devices 1 and 2, having no hole injection layer, similarly emitted white light, albeit at slightly lower light emission efficiency. These observations demonstrate that it was only the cis-compound obtained in synthesis example 4 and the trans-compound obtained in synthesis example 5 that were eligible white color EL luminescent sources. Furthermore, as shown in Table 1, the CIE coordinates of the white light emitted by these devices were very close to the CIE coordinate for pure white (0.33, 0.33).

### INDUSTRIAL APPLICABILITY

The organic EL device of the present invention is suitable for use, as a light source in the white light illumination device and also as a white light source in the organic EL display device.

### BRIEF DESCRIPTION OF DRAWINGS

FIG. 1 An ultraviolet-to-visible absorption spectrum and a photoluminescence spectrum of the cis-compound obtained in synthesis example 4 in a solution state and in a solid state.

FIG. 2 An ultraviolet-to-visible absorption spectrum and a photoluminescence spectrum of the trans-compound obtained in synthesis example 5 in a solution state and in a solid state.

FIG. 3 Schematics of organic EL devices 1 to 4.

FIG. 4 EL spectra of organic EL devices 1 to 4 under applied voltage.

FIG. 5 Current-voltage-luminance properties of organic EL device 1.

FIG. 6 Current-voltage-luminance properties of organic EL device 2.

FIG. 7 Current-voltage-luminance properties of organic EL device 3.

FIG. 8 Current-voltage-luminance properties of organic EL device 4.

FIG. 9 A photoluminescence spectrum of the cis-compound obtained in synthesis example 7 in a solution state.

FIG. 10 A photoluminescence spectrum of the trans-compound obtained in synthesis example 8 in a solution state.

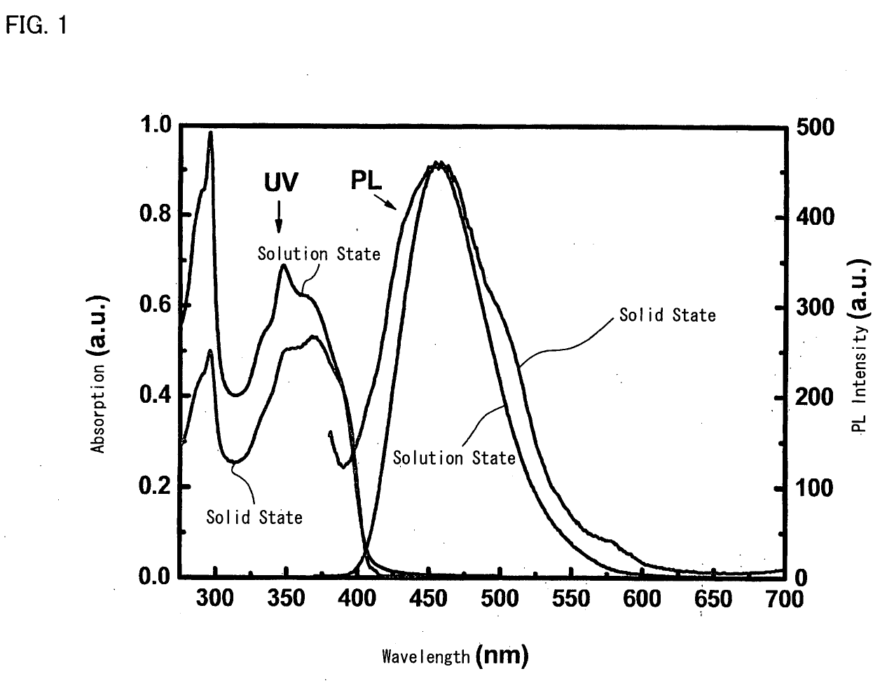

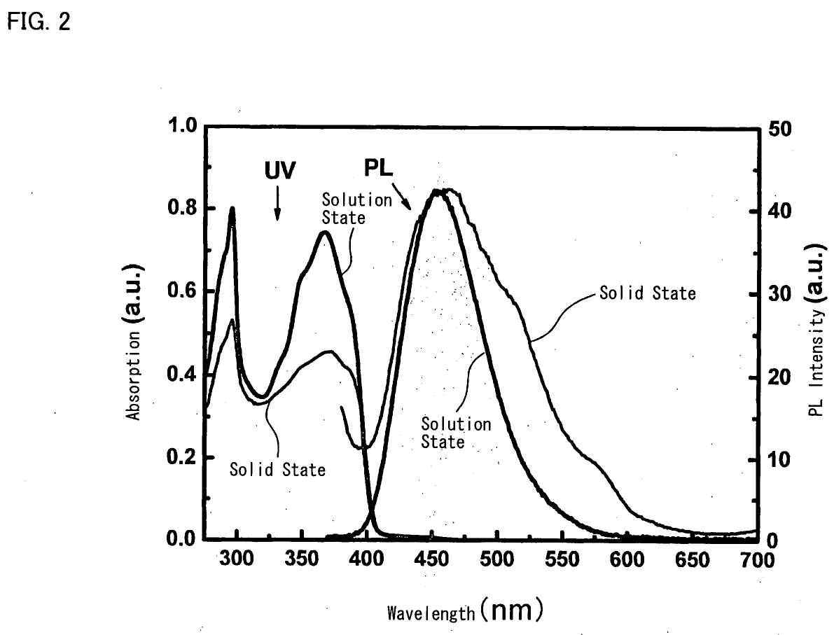

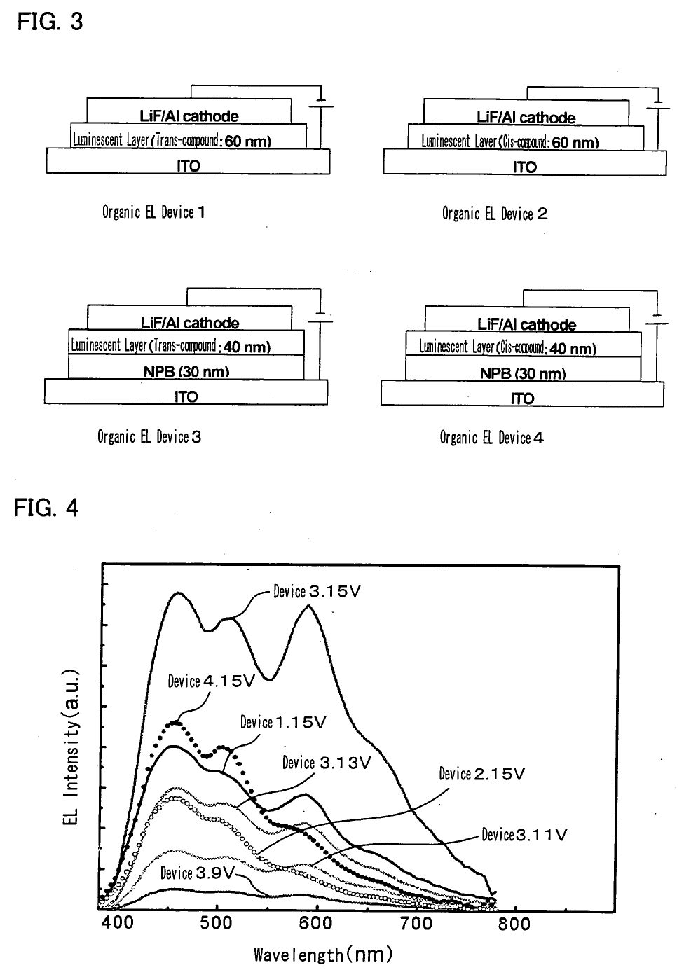

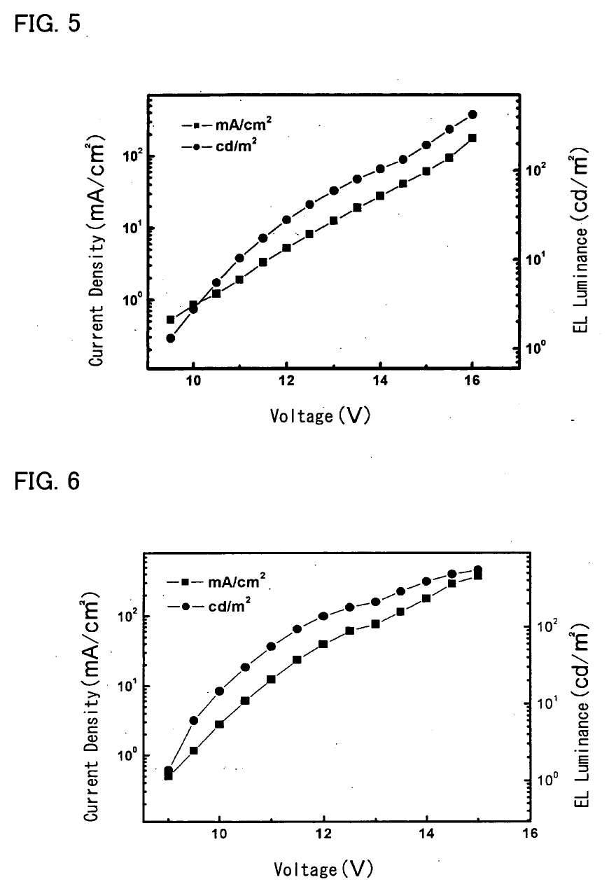

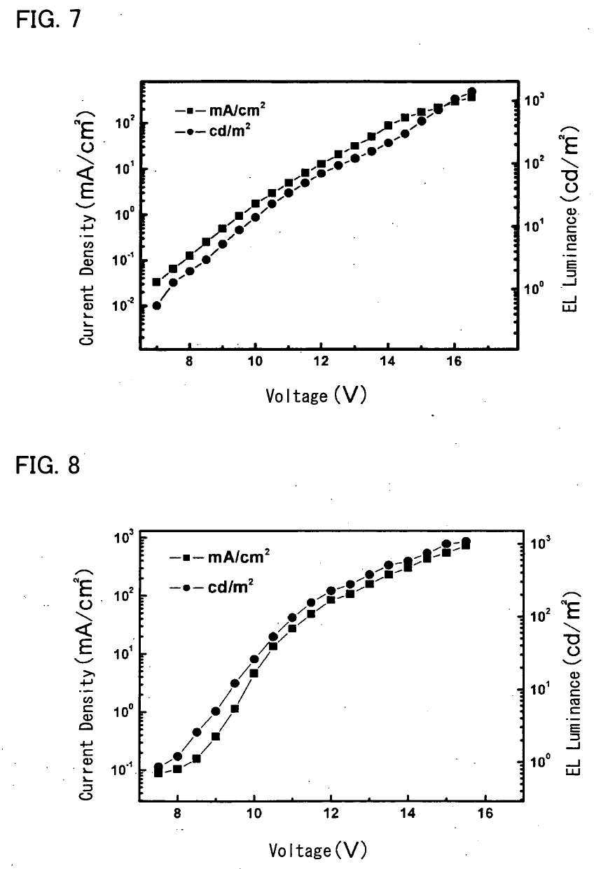

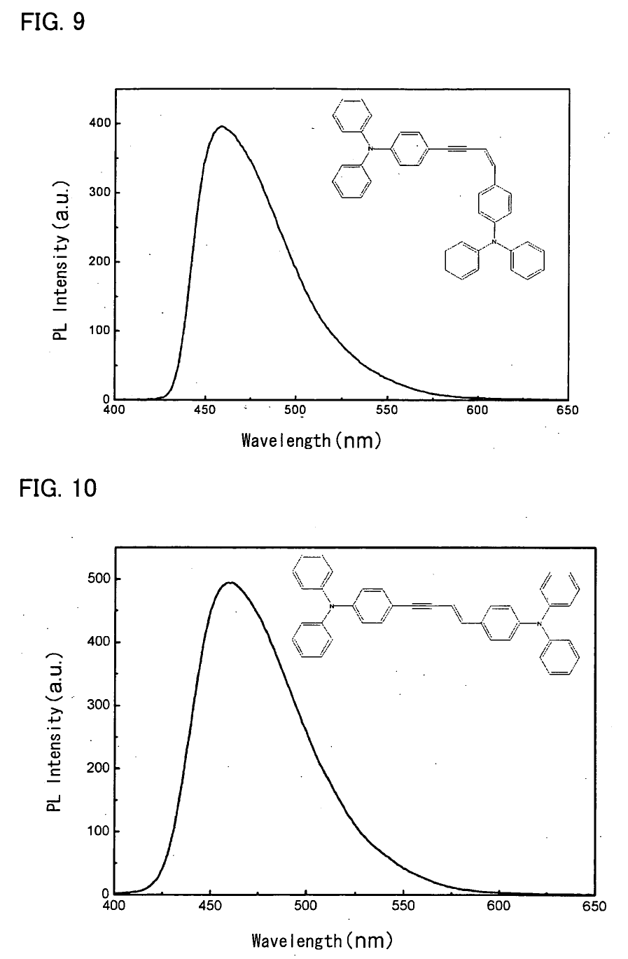

1. A compound having general formula (I): 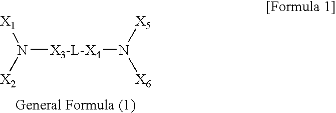
where each X₁, X₂, X₅, and X₆ is independently an (un)substituted aryl group or monovalent aromatic heterocyclic group; X₁ and X₂ may be bonded to form a ring; X₅ and X₆ may be bonded to form a ring; each of X₃ and X₄ is independently an (un)substituted arylene group or divalent aromatic heterocyclic group; and L is either

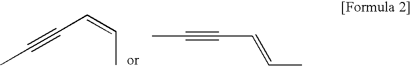

2. The compound set forth in claim 1, having general formula (II): 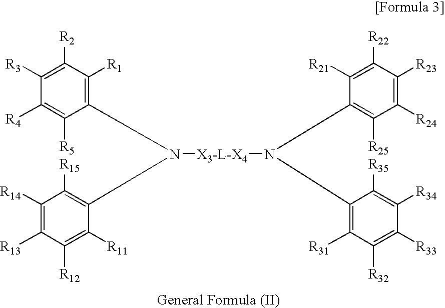
where each of R₁, R₂, R₃, R₄, R₅, R₁₁, R₁₂, R₁₃, R₁₄, R₁₅, R₂₁, R₂₂, R₂₃, R₂₄, R₂₅, R₃₁, R₃₂, R₃₃, R₃₄, and R₃₅ is independently a hydrogen atom, an (un)substituted alkyl group, an alkoxy group, or an aryl group; R₅ and R₁₅ may be bonded; R₂₅ and R₃₅ may be bonded; and L, X₃, and X₄ have the same definition as in general formula (I).

3. The compound set forth in claim 1, wherein each of X₃ and X₄ is independently an (un)substituted phenylene group.

4. The compound set forth in claim 1, having general formula (III): 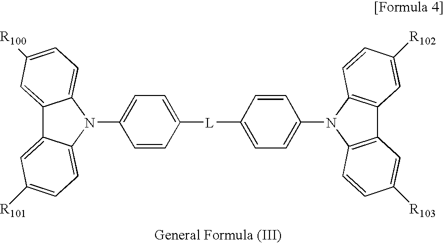
where each of R₁₀₀, R₁₀₁, R₁₀₂, and R₁₀₃ is independently an (un)substituted alkyl group, an alkoxy group, or an aryl group; and L has the same definition as in general formula (I).

5. An organic EL device, comprising: a pair of electrodes and a luminescent layer, sandwiched between the pair of electrodes, containing a luminescent material containing the compound set forth in claim 1.

6. An organic EL device, comprising: a pair of electrodes and a luminescent layer, sandwiched between the pair of electrodes, containing a luminescent material consists of the compound set forth in claim 1.

7. The organic EL device set forth in claim 5, further comprising a hole injection layer between the pair of electrodes.

8. The organic EL device set forth in claim 5, wherein the luminescent layer emits white light when a voltage is applied across the pair of electrodes.

9. A method of using the organic EL device set forth in claim 5 as a white light emitter.

10. The method set forth in claim 9, comprising the step of applying a voltage across the pair of electrodes to cause the luminescent layer to emit white light.

11. A method of manufacturing an organic EL device including a pair of electrodes and a luminescent layer, sandwiched between the pair of electrodes, containing a luminescent material, wherein the compound set forth in claim 1 is used as the luminescent material.

12. A method of using the compound set forth in claim 1 as a luminescent material in an organic EL device including a luminescent layer containing the luminescent material between a pair of electrodes.
